(19) United States
(12) Patent Application Publication
(10) Pub. No.: US 2018/0155311 A1
(43) Pub. Date: Jun. 7, 2018

(54) SEPARATION OF CHIRAL ISOMERS BY SFC

(71) Applicant: DSM IP ASSETS B.V., Heerlen (NL)

(72) Inventors: Dominik BURGER, Kaiseraugst (CH); Thomas NETSCHER, Kaiseraugst (CH); Gerhard SCHIEFER, Kaiseraugst (CH)

(21) Appl. No.: 15/575,994

(22) PCT Filed: May 23, 2016
(86) PCT No.: PCT/EP2016/061554

§ 371 Date: Nov. 21, 2017

(30) Foreign Application Priority Data
May 26, 2015 (EP) 15169171.4

(51) Int. Cl. C07D 311/72; B01D 15/40; B01D 15/38; A23K 20/10; A23L 33/10

(52) U.S. Cl. CPC C07D 311/72; B01D 15/40; A23V 2002/00; A23K 20/10; A23L 33/10; B01D 15/3833

(57) ABSTRACT

The present invention relates to the field of separating chiral isomers from each other. Particularly, it relates to the field of separating of chiral isomers of chromane or chromene compounds, particularly tocopherols, 3,4-dehydro-tocopherols and tocotrienols, as well as the protected forms thereof. It has been found that the use of supercritical carbon dioxide as mobile phase combined with the very specific chiral phase as stationary phase leads to a very efficient separation of the individual chiral isomers. As the method is very efficient and fast combined with advantageous in view of ecology it is of big industrial interest.

### TECHNICAL FIELD

The present invention relates to the field of separating chiral isomers from each other. Particularly, it relates to the field of separating of chiral isomers of chromane and chromene compounds.

### BACKGROUND OF THE INVENTION

The presence of chiral centers in a molecule often leads to different chiral isomers. The larger the number of chiral centers in a molecule the larger the number of different isomers is. In the synthesis of such chiral molecules normally a mixture of chiral isomers is formed. However, very often, it is desirable to separate chiral compounds from each other, for example as they have different properties.

Chromane compounds represent an important class of chiral natural products and bioactive compounds. An important class of chromane compounds are vitamin E and its esters. Often vitamin E is commercialized in the form of its esters because the latter show an enhanced stability.

On the one hand the typical technical synthesis of vitamin E leads to mixtures of isomers. On the other hand higher bioactivity (biopotency) has been shown to occur in general by tocopherols having the R-configuration at the chiral center situated next to the ether atom in the ring of the molecule (indicated by * in the formulas used later on in the present document) (i.e. 2R-configuration), as compared to the corresponding isomers having S-configuration. Particularly active are the isomers of tocopherols having the natural configuration at all chiral centers, for example (R,R,R)-tocopherols, as has been disclosed for example by H. Weiser et al. in J. Nutr. 1996, 126(10), 2539-49 This leads to a strong desire for an efficient process for separating the isomers. Hence, the separation relates not only to the separation of the structurally different tocopherols but also the different chiral isomers of the same chemical structure, i.e. within the same tocopherol.

Whereas the separation of the different tocopherols, i.e. α-tocopherol, β-tocopherol, γ-tocopherol and δ-tocopherol or of different tocotrienols, respectively the protected form thereof, has been achieved already long time ago by conventional chromatographic techniques, the separation of the chiral isomers is a much more challenging goal. Due to the chemical similarity and the same chemical structure of the isomers they are extremely difficult to separate by chromatographic techniques.

Chromatographic separation of chiral compounds has been found to be an adequate method for the separation of certain chiral substances as is disclosed by S. K. Jensen in Vitamins and Hormones 2007, Vol. 76, 281-308. Particularly suited for industrial chromatographic separation processes is Simulated Moving Bed (SMB) chromatography as this leads to enhanced separation efficiency and reduced amount of eluent necessary for the separation.

WO 2012/152779 A1 discloses a process of separation of chiral chromane or chromene compounds involving a chromatographic separation step by means of a chiral phase and an isomerization step.

Supercritical fluids show physical properties which position them between liquids and gases. Like gases they are easily compressible and properties like density and viscosity can be modified by pressure and temperature changes. Supercritical carbon dioxide is used on a large industrial scale for the decaffeination of green coffee beans or the extraction of hops for beer production. It has been also been proposed by EP 1 000 940 A1 to use supercritical carbon dioxide as reaction medium in the synthesis of tocopherol.

Furthermore, due to its highly advantageous properties of low viscosity and high diffusion rates, supercritical carbon dioxide has found application in the supercritical fluid chromatography (SFC) for separating chemical compounds. For example EP 1 122 250 A1 discloses the chromatographic separation of structural isomers of tocopherols (α-tocopherol, β-tocopherol, γ-tocopherol, δ-tocopherol) and of tocotrienols (α-tocotrienol, β-tocotrienol, γ-tocotrienol and δ-tocotrienol) from each other by supercritical carbon dioxide as mobile phase and silica gel or C18 reversed phase silica gel as stationary phase. However, no separation of the chiral isomers having the same chemical structure has been achieved by this method.

Hence, there remains the desire to be able to separate chiral isomers of the same chemical structure of tocopherols respectively their protected forms.

### SUMMARY OF THE INVENTION

The problem to be solved by present invention is to offer an easy and fast separation method of the chiral isomers of chromane or chromene compounds, respectively the protected forms thereof.

Surprisingly, it has been found that the process according to claim 1 is able to solve this problem.

It has been found that the use of supercritical carbon dioxide as mobile phase combined with the very specific chiral phase as stationary phase leads to a very efficient separation of the individual chiral isomers.

This method does not only allow separation of the chromane or chromene compounds from each other but also the separation of the individual chiral isomers.

Particularly, it has been shown that the biologically highly active (2R, 4′R, 8′R)-tocopherols could be isolated from the (all-rac)-tocopherol, respectively 2-ambo-tocopherol.

However, the process is not only limited to the isolation of a single chiral isomer, but also other chiral isomers can be easily isolated individually of a mixture of chiral isomers. It has been found, that the majority of the peaks, corresponding to the individual isomers, are particularly well baseline separated, allowing an efficient separation of said chiral isomer in high isomeric purity.

The separation is not only very advantageous in view of separation efficiency but also exhibits an exceptionally high separation speed. It has been shown that this method is able to separate the chiral isomers in less than 5 minutes in an analytical scale and less than 9 minutes in semi-preparative scale.

This high separation speed is very advantageous not only in view of analytical use, but particularly also in view of preparative separation.

The use of supercritical carbon dioxide as mobile phase eliminates or reduces to a large extent the use of organic solvents in the separation. This is very advantageous in view of ecology and working safety.

This process offers, therefore, a unique possibility to the isolation of chiral isomers of high biological activities stemming from synthetic origin and is, hence, highly interesting particularly in the fields of food, feed, food supplements, feed supplements and pharmaceutical compositions.

Further aspects of the invention are subject of further independent claims. Particularly preferred embodiments are subject of dependent claims.

### DETAILED DESCRIPTION OF THE INVENTION

The present invention relates in a first aspect to a process of separating chiral isomers of chromane or chromene compounds of compounds of formula (I-A) or (I-B)

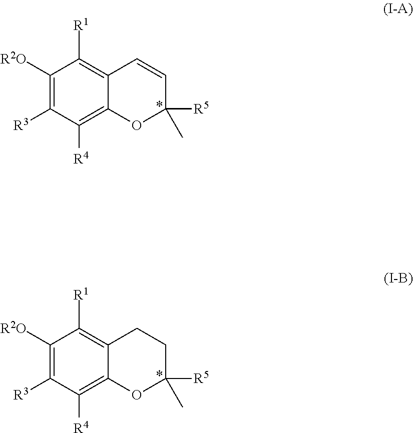

wherein R¹, R³ and R⁴ are independently from each other hydrogen or methyl groups;
R² represents hydrogen or a phenol protection group;
R⁵ represents either a linear or branched completely saturated C₆₋₂₅-alkyl group or a linear or branched C₆₋₂₅-alkyl group comprising at least one carbon-carbon double bond;
and wherein * represents a chiral center;
   comprising the steps of
   a) providing a mixture of isomers of formula (I-A) or (I-B) having different chiral configuration at the chiral centers represented by * in formula (I-A) or (I-B);
   b) chiral chromatographic separation of the mixture of isomers of step a) by means of supercritical fluid chromatography with supercritical carbon dioxide as a mobile phase and an amylose tris(3,5-dimethylphenyl-carbamate) coated or immobilized on a silica support as a chiral stationary phase (CSP).

The term “independently from each other” in this document means, in the context of substituents, moieties, or groups, that identically designated substituents, moieties, or groups can occur simultaneously with a different meaning in the same molecule.

In the present document any dotted line represents the bond by which a substituent is bound to the rest of a molecule.

A “C_{x-y}-alkyl”, resp. “C_{x-y}-acyl” group, is an alkyl resp. an acyl group comprising x to y carbon atoms, i.e. for example an C₁₋₃-alkyl group, is an alkyl group comprising 1 to 3 carbon atoms. The alkyl resp. the acyl group can be linear or branched. For example —CH(CH₃)—CH₂-CH₃ is considered as a C₄-alkyl group.

The “pKₐ” is commonly known as negative decadic logarithm of the acid dissociation constant (pKₐ=−log₁₀ Kₐ). When the organic acid has several protons the pKₐ relates to the dissociation of the first proton (Kₐ₁). The pKₐ values indicated are at room temperature. The person skilled in the art knows that the acidities of certain acids are measured in adequate solvents and may vary upon individual measurements or due to the fact the determination of the pKₐ has been measured in different solvents and, hence, different pKₐ values can be found for a specific acid. Hence, in a critical case, where for an acid different pKₐ values can be found in literature of which at least one is in the pKₐ range indicated by the present document—whereas other values are found being outside of said range—it is defined that such an acid is considered to be in the range of pKₐ values.

In the present document the term “isomerized” or “isomerization” relates to a change in chirality. Therefore, structural isomerization leading to another connectivity of atoms is not meant by this term. Furthermore, this term, for this document, also excludes cis/trans isomerization.

In the present document any single dotted line represents the bond by which a substituent is bound to the rest of a molecule.

The chirality of an individual chiral carbon center is indicated by the label R or S according to the rules defined by R. S. Cahn, C. K. Ingold and V. Prelog.

This R/S-concept and rules for the determination of the absolute configuration in stereochemistry is known to the person skilled in the art.

The term “isomeric purity” (“IP”) describes in this document the amount of an isomer having a specific chirality in view of the amount of all isomers in a sample. For example, the isomeric purity of (2R, 4′R, 8′R)-α-tocopherol (IP₍2R,4′R,8′R)) in a sample is given by

$${IP}_{( 2 R , {4}^{’} R , {8}^{’} R )} = \frac{[ ( 2 R , {4}^{’} R , {8}^{’} R ) ]}{\begin{matrix}[ ( 2 R , {4}^{’} R , {8}^{’} R ) ] + [ ( 2 R , {4}^{’} S , {8}^{’} R ) ] + [ ( 2 R , {4}^{’} R , {8}^{’} S ) ] + \\ [ ( 2 R , {4}^{’} S , {8}^{’} S ) ] + [ ( 2 S , {4}^{’} R , {8}^{’} R ) ] + [ ( 2 S , {4}^{’} S , {8}^{’} R ) ] + \\ [ ( 2 S , {4}^{’} R , {8}^{’} S ) ] + [ ( 2 S , {4}^{’} S , {8}^{’} S ) ]\end{matrix}}$$

In this (above) formula [(2X, 4′Y, 8′Z)] stands for the amount (in mol) of the individual isomer having the given chirality at chiral centers at the 2, 4′ and 8′ position of the α-tocopherol (X═R or S, Y═R or S and Z═R or S). The isomeric purity is given in %.

Said process allows separating chiral isomers of formula (I-A) or (I-B). Formula (I-A) or (I-B) has different substituents, i.e. R¹, R², R³ , R⁴ and R⁵.

The residue R⁵ represents either a linear or branched completely saturated C₆₋₂₅-alkyl group or a linear or branched C₆₋₂₅-alkyl group comprising at least one carbon-carbon double bond.

Preferably the group R⁵ is of formula (III).

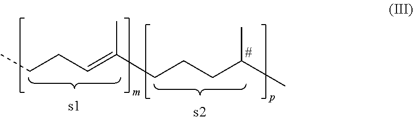

In formula (III) m and p stand independently from each other for a value of 0 to 5 provided that the sum of m and p is 1 to 5. Furthermore, the substructures in formula (III) represented by s1 and s2 can be in any sequence. The dotted line represents the bond by which the substituent of formula (III) is bound to the rest of the compound of formula (I-A) or formula (I-B). Furthermore, # represents a chiral center, obviously except in case where said center is linked to two methyl groups.

It is preferred that group R⁵ is of formula (III-x).

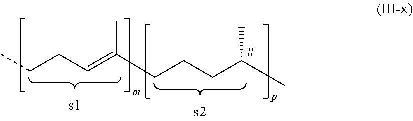

As mentioned above the substructures in formula (III) represented by s1 s2 can be in any sequence. It is, therefore, obvious that in case that the terminal group is having the substructure s2, this terminal substructure has no chiral center.

The double bond(s) of formula (III) or (III-x) can have either E or Z configuration. Preferably the double bond(s) is/are in E-configuration, most preferred all double bonds in formula (III) or (III-x) are in the E-configuration.

In one preferred embodiment m stands for 3 and p for 0.

In another preferred embodiment p stands for 3 and m for 0.

Therefore, R⁵ is preferably of formula (III-A), particularly (III-ARR), or (III-B).

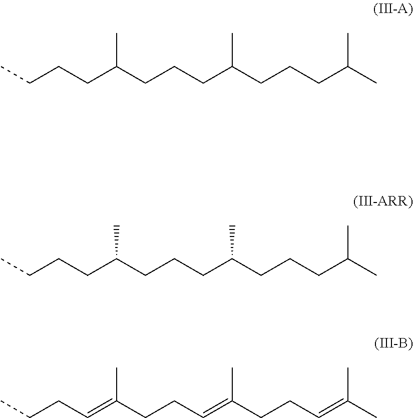

Preferred are the following combinations of R¹, R³ and R⁴:

R¹═R³═R⁴=CH₃

or

R¹═R⁴═CH₃, R³═H

or

R¹═H, R³═R⁴═CH₃

or

R¹═R³═H, R⁴═CH₃.

More preferred is that R¹═R³═R⁴═CH₃.

Preferably the chiral isomers of formula (I-B) are the isomers selected from the group consisting of
α-tocopherol (R¹═R³═R⁴═CH₃, R²═H, R⁵═(III-A));
β-tocopherol (R¹═R⁴═CH₃, R³═H, R²═H, R⁵═(III-A)),
γ-tocopherol (R¹═H, R³═R⁴═CH₃, R²═H, R⁵═(III-A)),
δ-tocopherol (R¹═R³═H, R⁴═CH₃, R²═H, R⁵═(III-A));
α-tocotrienol (R¹═R³═R⁴═CH₃, R²═H, R⁵═(III -B)),
β-tocotrienol (R¹═R⁴═CH₃, R³═H, R²═H, R⁵═(III-B)),
γ-tocotrienol (R¹═H, R³═R⁴═CH₃, R²═H, R⁵═(III-B)),
δ-tocotrienol (R¹═R³═H, R⁴═CH₃, R²═H, R⁵═(III-B))

and the protected forms, particularly the esters, preferably the acetates (R²═COCH₃), thereof.

More preferably the chiral isomers of formula (I-B) are the isomers selected from the group consisting of α-tocopherol, γ-tocopherol, α-tocotrienol and γ-tocotrienol, particularly α-tocopherol or α-tocotrienol, and the protected forms, particularly the esters, preferably the acetates (R²═COCH₃), thereof.

Preferably the chiral isomers of formula (I-A) are the isomers selected from the group consisting of
3,4-dehydro-α-tocopherol (R¹═R³═R⁴═CH₃, R²═H, R⁵═(III-A));
3,4-dehydro-β-tocopherol (R¹═R⁴═CH₃, R³═H, R²═H, R⁵═(III-A)),
3,4-dehydro-γ-tocopherol (R¹═H, R³═R⁴═CH₃, R²═H, R⁵═(III-A)),
3,4-dehydro-δ-tocopherol (R¹═R³═H, R⁴═CH₃, R²═H, R⁵═(III-A));
3,4-dehydro-α-tocotrienol (R¹═R³═R⁴═CH₃, R²═H, R⁵═(III-B)),
3,4-dehydro-β-tocotrienol (R¹═R⁴═CH₃, R³═H, R²═H, R⁵═(III-B)),
3,4-dehydro-γ-tocotrienol (R¹═H, R³═R⁴═CH₃, R²═H, R⁵═(III-B)),
3,4-dehydro-δ-tocotrienol (R¹═R³═H, R⁴═CH₃, R²═H, R⁵═(III-B))
and the protected forms, particularly the esters, preferably the acetates (R²═COCH₃), thereof.

More preferably the chiral isomers of formula (I-A) are the isomers selected from the group consisting of 3,4-dehydro-α-tocopherol and 3,4-dehydro-α-tocotrienol, particularly 3,4-dehydro-α-tocopherol, and the protected forms, particularly the esters, preferably the acetates (R²═COCH₃), thereof.

R² represents either hydrogen or a phenol protection group.

A phenol protection group is a group which protects the phenolic group (OH in formula (I-A) or (I-B)) and can be deprotected easily, i.e. by state-of-the-art methods, to the phenolic group again.

The phenol protection group forms with the rest of the molecule a chemical functionality which is particularly selected from the group consisting of ester, ether or acetal. The protection group can be easily removed by standard methods known to the person skilled in the art.

In case where the phenol protection group forms with the rest of the molecule an ether, the substituent R² is particularly a linear or branched C₁₋₁₀-alkyl or cycloalkyl or aralkyl group. Preferably the substituent R² is a benzyl group or a substituted benzyl group, particularly preferred a benzyl group.

In case where the phenol protection group forms with the rest of the molecule an ester, the ester is an ester of an organic or inorganic acid.

If the ester is an ester of an organic acid, the organic acid can be a monocarboxylic acid or a polycarboxylic acid, i.e. an acid having two or more COOH-groups. Polycarboxylic acids are preferably malonic acid, succinic acid, glutaric acid, adipic acid, maleic acid or fumaric acid.

Preferably the organic acid is a monocarboxylic acid.

Hence, the substituent R² is preferably an acyl group. The acyl group is particularly a C₁₋₇-acyl, preferably acetyl, trifluoroacetyl, propionyl or benzoyl group, or a substituted benzoyl group.

If the ester is an ester of an inorganic acid, the inorganic acid is preferably nitric acid or a polyprotic acid, i.e. an acid able to donate more than one proton per acid molecule, particularly selected from the group consisting of phosphoric acid, pyrophosphoric acid, phosphorous acid, sulphuric acid and sulphurous acid.

In case where the phenol protection group forms with the rest of the molecule an acetal, the substituent R² is preferably

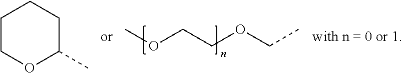

Hence, the acetals formed so are preferably methoxymethyl ether (MOM-ether), β-methoxyethoxymethyl ether (MEM-ether) or tetrahydropyranyl ether (THP-ether). The protection group can easily be removed by acid.

The protecting group is introduced by reaction of the corresponding molecule having an R² being H with a protecting agent.

The protecting agents leading to the corresponding phenol protection groups are known to the person skilled in the art, as well as the chemical process and conditions for this reaction. If, for example, the phenol protection group forms with the rest of the molecule an ester, the suitable protecting agent is for example an acid, an anhydride or an acyl halide.

In the case that an ester is formed by the above reaction with the protecting agent, and that said ester is an ester of an organic polycarboxylic acid or an inorganic polyprotic acid, not necessarily all acid groups are esterified to qualify as protected in the sense of this document. Preferable esters of inorganic polyprotic acids are phosphates.

It is preferred that the protection group R² is a benzoyl group or a C₁₋₄-acyl group, particularly acetyl or trifluoroacetyl group. The molecules in which R² represents an acyl group, particularly an acetyl group, can be easily prepared from the corresponding unprotected molecule by esterification, respectively the phenolic compound can be obtained from the corresponding ester by ester hydrolysis.

It is most preferred that R² is H.

In a preferred embodiment compounds of formula(I-A) or (I-B) are of formula (I-A-I) or (I-B-I)

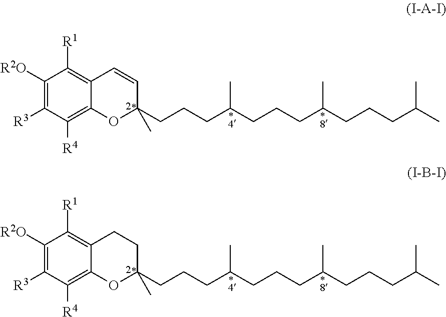

The formula (I-A-I) or (I-B-I) has 3 chiral centers. These chiral centers are marked by the symbol * in the formulae of this document. The chiral centers are located at the positions 2, 4′ and 8′.

This leads to 8 different chiral isomers for compounds of formula (I-A-I) or (I-B-I) a given specific combination of R¹, R², R³ and R⁴ .

The present process is suited for separating chiral isomers having different chirality at said chiral centers. Particularly, these chiral isomers are structurally identical except of their chirality.

Said process comprises the step a) of providing a mixture of isomers of formula (I-A) or (I-B) having different chiral configuration at the chiral centers represented by * in formula (I-A) or (I-B). Such a mixture can be a result of a synthetic synthesis of said isomers or of a reaction involving a precursor of said isomers. The precursors can be of synthetic or biological origin. Furthermore, it is possible that such a mixture is of biological origin.

Industrially most important are mixtures being a result of a chemical synthesis.

The compounds of formula (I-A) or (I-B) can be prepared by known methods.

The compounds of formula (I-A) can be obtained for example by the reduction of formula (I-aa), particularly by sodium boranate and subsequent elimination of water, as disclosed by Kabbe and Heitzer, Synthesis 1978, 888-889.

A preferred way of synthesizing compounds of formula (I-aa) is from the corresponding 2-acetyl-methylhydroquinone, 2-acetyl-dimethylhydroquinone resp. 2-acetyl-trimethylhydroquinone of formula (XX) with R²═H, or the corresponding compound of formula (XX) with R² being a phenol protecting group, and the ketone of formula (XXI), particularly farnesylacetone, in the presence of a base, particular in the presence of pyrrolidine, as disclosed in detail by Kabbe and Heitzer, Synthesis 1978, 888-889.

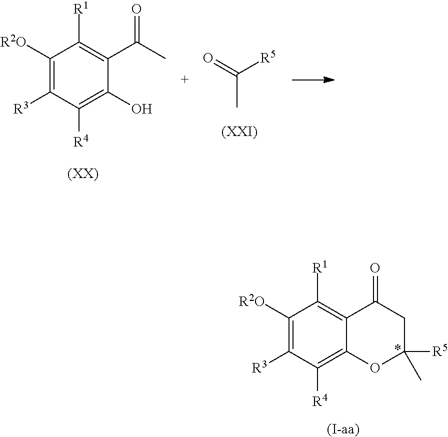

Compound of formula (I-B) and, particularly compounds of formula (I-B-I), can be synthesized from the corresponding methyl-, dimethyl- respectively trimethylhydoquinone of formula (X) and the corresponding alcohol, particularly of formula (XI-A) respectively (XI-B), in a known manner (Ullmann's Encyclopedia of Industrial Chemistry, Release 2010, 7^{th} Edition, “Vitamins”, page 44-46)

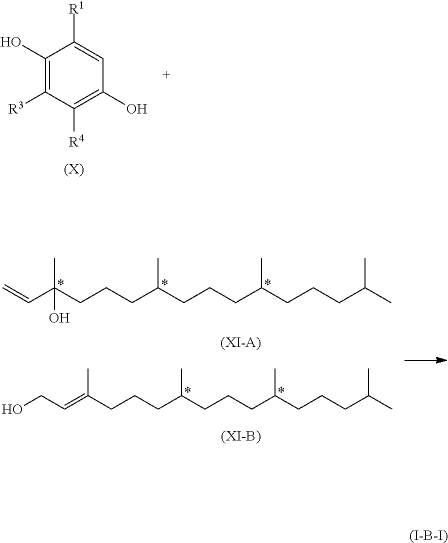

Said reactions are not stereospecific and, hence, a mixture of isomers of formula (I-A) or (I-B) of R- and S- configuration at the chiral center marked by * in the 2 position is formed. Typically racemic mixtures of about 50% S- and 50% R-isomers are formed at the chiral center in 2 position of formula (I-A) or (I-B).

Phytol, the alcohol of formula (XI-B), is typically an isomeric mixture of 4 isomers ((R,R)- (R,S)-, (S,R)- and (S,S)-isomer) being synthesized according the traditional methods.

Using an isomeric mixture of phytol, respectively isophytol, leads to a mixture of 8 isomers of formula (I-B-I ) having mixed configuration at all chiral centers (position 2, 4′ and 8′).

Mixtures of isomers of formula (I-A) or (I-B) having mixed configuration at all chiral centers are called “(all-rac)” in the present document. Hence, such a mixture in formula (I-B-I ) having mixed configuration at all chiral centers (position 2, 4′ and 8′) is called “(all-rac)-tocopherol” in the case where R²═H. The term “(all-rac)-α-tocopherol” means, therefore, a mixture of 8 isomers of α-tocopherol.

Hence, in one embodiment it is preferred that the mixture of chiral isomers of formula (I-B) is (all-rac)-tocopherol, particularly (all-rac)-α-tocopherol.

In a further preferred embodiment, the mixture of chiral isomers of formula (I-B) is (all-rac)-tocopheryl acetate, particularly (all-rac)-α-tocopheryl acetate.

In a further preferred embodiment, the mixture of chiral isomers of formula (I-A) is (all-rac)-3,4-dehydro-tocopherol, particularly (all-rac)-3,4-dehydro-α-tocopherol, or the acetate thereof.

In contrast to this, natural phytol consists only of the (R,R)-isomer and hence, is isomerically pure.

Therefore, in one preferred embodiment the compound of formula (I-B-I ) is prepared from natural phytol. However, as natural phytol, respectively isophytol, is commercially available only in rather small amounts and is rather expensive, the potential of using natural phytol, respectively isophytol, for industrial scale synthesis of tocopherols is rather limited.

However, new developments enable synthesizing phytol in a preferential formation of a single isomer. For example WO 2006/066863 A1 or WO 2012/171969 A1, the entire content of which is hereby incorporated by reference, disclose methods of asymmetric hydrogenation of alkenes using chiral iridium complexes. It has been found that using this method leads to the desired isomer of the chiral hydrogenation products of the corresponding alkene in selectivity which then can be converted further chemically to the desired isomer of phytol respectively isophytol. Phytol, respectively isophytol, then can be transformed by further known chemical transformations finally to the desired isomer of tocopherol.

Therefore, in another preferred embodiment the compound of formula (I-B-I ) is prepared from isophytol being obtained in a multistep reaction comprising an asymmetrical hydrogenation of alkene in the presence of a chiral iridium complex. Using an isomerically pure phytol, respectively isophytol, leads to a mixture of 2 isomers having mixed configuration at the chiral center of position 2 in formula (I-B-I ).

A mixture having mixed configuration (only) at the chiral center of position 2 is called “ambo” resp. “(2-ambo-)” in the present document. Hence, such a mixture of (2R, 4′R, 8′R)-tocopherol and (2S, 4′R, 8′R)-tocopherol is also known as (2-ambo)-tocopherol in the case where R²═H. Therefore, (2-ambo)-α-tocopherol is a mixture of (2R, 4′R, 8′R)-α-tocopherol and (2S, 4′R, 8′R)-α-tocopherol.

Hence, in another preferred embodiment the mixture of chiral isomers of formula (I-B) is (2-ambo)-tocopherol, particularly (2-ambo)-α-tocopherol.

In a further preferred embodiment the mixture of chiral isomers of formula (I-B) is (2-ambo)-tocopheryl acetate, particularly (2-ambo)-α-tocopheryl acetate.

Tocotrienols and 3,4-dehydrotocotrienols have only one chiral carbon, i.e. the carbon center at the 2 position. Hence, mixture of (2R)-tocotrienol and (2S)-tocotrienols is called “(rac)-tocotrienol” and a mixture of (2R)-3,4-dehydro-tocotrienol and (2S)-3,4-dehydro-tocotrienols is called “(rac)-3,4-dehydrotocotrienol”.

In a further preferred embodiment the mixture of chiral isomers of formula (I-B) is “(rac)-tocotrienol, particularly “(rac)-α-tocotrienol, or the acetate thereof. In a further preferred embodiment the mixture of chiral isomers of formula (I-A) is (2-ambo)-3,4-dehydro-tocopherol, particularly (2-ambo)-3,4-dehydro-α-tocopherol, or the acetate thereof.

Said process further comprises the step b) of chiral chromatographic separation of the mixture of isomers of formula (I-A) or (I-B) by means of supercritical fluid chromatography with supercritical carbon dioxide as a mobile phase and an amylose tris(3,5-dimethylphenylcarbamate) coated or immobilized on a silica support as a chiral stationary phase (CSP).

Supercritical carbon dioxide is a fluid state of carbon dioxide where it is held at or above its critical temperature and critical pressure. For carbon dioxide the critical temperature (T_{c}) is 31.3° C. and the critical pressure (p_{c}) is 7.39 MPa. Supercritical carbon dioxide is used as mobile phase for the supercritical fluid chromatography.

As chiral stationary phase (CSP) amylose tris(3,5-dimethylphenyl-carbamate) coated or immobilized on a silica support is used.

Amylose tris(3,5-dimethylphenylcarbamate) is an amylose which is modified by a chemical reaction, for example shown in U.S. Pat. No. 4,861,872, so that 80% to 100% of the H of the hydroxyl groups of the amylose are converted into 3,5-dimethylphenylcarbamate groups yielding a chemical formula

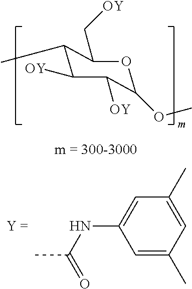

The chiral stationary phase can be prepared by attaching this chiral compound to the surface of silica, an achiral solid support, particularly on silica gel. The chiral compound may be immobilized or form a coating on the silica support. The chiral compound can be adsorbed or chemically bound to the support. Preferably the chiral compound is chemically bound to the support.

Such chiral phases are described in U.S. Pat. No. 4,861,872 and Pure Appl. Chem., Vol. 79, No. 9, 2007, 1561-1573, the entire content of which is hereby incorporated by reference.

Particularly suitable are the chiral phases which are commercially available Chiralpak® IA and IA-3 and AD and AD-H and AD-3 from Daicel Chemical Industries Ltd., Japan.

It has been found that other chiral stationary phase (CSP) based on other polysaccharides such as cellulose are not suited, respectively much less suited. Similarly also different derivatisations (e.g. amylose tris((S)-α-methylbenzyl-carbamate) of the amylose showed no, respectively remarkably lower separation. For example the chiral stationary phases Chiralpak® IB or IC or OZ or OJ-H or AS-H showed no respectively insufficient separation of the chiral isomers.

The particle size of the chiral phase is in one embodiment smaller than 25 micrometer, particularly between 3 and 25 micrometer, preferably between 5 and 25 micrometer, more preferably between 5 and 15 micrometer.

Another embodiment the particle size of the chiral phase is larger than 25 micrometer, particularly between 50 and 70 micrometer. It is preferable as by using such larger particle sizes the pressure to be applied can be lower. These larger particle sizes are particularly suited for preparative separations.

It has been shown that the chiral stationary phases Chiralpak® AD-H and Chiralpak® AD-3 from Daicel Chemical Industries Ltd., Japan, are particularly suited for the purpose of this invention.

It has been shown that it is preferable to add certain solvents as modifiers to the mobile phase. Particularly it has been found very advantageous that the mobile phase comprises an alcohol, particularly an alcohol being selected from the group consisting of methanol, ethanol, 1-propanol, 2-propanol, 1-butanol, 2-butanol, 2-methyl-1-propanol and 2-methyl-2-propanol. Preferred are methanol and ethanol, most preferred methanol.

By adding these solvents to the mobile phase it has been observed that the selectivity of the separation is significantly improved. This is explained by the increase of the polarity of the supercritical mobile phase by adding the alcohol.

It has been observed that the alcohol is preferably used in a volume ratio of 1 to 30%, particularly of 1 to 25%, preferably of 5 to 20%, more preferably of 8 to 15%, relative to the supercritical carbon dioxide.

By adding other polar solvents such as acetonitrile, ethers (e.g. tert.-butylmethylether), esters (e.g. ethyl acetate), ketones (e.g. acetone), halogenated hydrocarbons (e.g. chloroform or dichloromethane) or water to the mobile phase this beneficial effect has not been observed.

In certain cases, the mobile phase can comprise also amines, particularly a secondary amine selected from the group consisting of dimethylamine, diethylamine and dipropylamine. Typically those amines are added in small amounts.

Furthermore, the mobile phase can also comprise at least an organic acid with a pKₐ of less than 6.0, particularly between 0.5 and 6.0, preferably between 3.0 and 6.0, particularly acetic acid.

Examples for organic acids having with a pKₐ of between 3.0 and 6.0, are particularly citric acid, phthalic acid, terephthalic acid, succinic acid, cinnamic acid, formic acid, lactic acid, acetic acid, ascorbic acid, benzoic acid, butanoic acid, propanoic acid and octanoic acid.

Acids having with a pKₐ of less than 6.0 are those mentioned above as well as acids such as sulphonic acids or halogenated acids are trifluoroacetic acid, trichloroacetic acid, p-toluenesulphonic acid, benzenesulfonic acid, dodecylbenzenesulfonic acid, methanesulphonic acid, trifluoromethanesulfonic acid and nonafluorobutanesulphonic acid.

It is preferred that the amount of supercritical carbon dioxide in the mobile phase is more than 75% by volume, preferably more than 85%, particularly 90% or more, by volume.

The chromatographic separation is preferably done at a temperature in the range of between 31.3° C. and 80° C., preferably in the range of between 32° C. and 50° C., particularly between 35° C. and 45° C.

It has been, furthermore, observed that the chromatographic separation is done at a pressure in the range of between 73.9 bar (7.39 MPa) to 200 bar, particularly between 74 to 150 bar, preferably between 74 and 100 bar, more preferably between 74 and 90 bar.

Best results of separation have been observed at temperature near the critical temperature (31.3° C.) and near the critical pressure (73.9 bar) of carbon dioxide.

The equipment for chiral chromatographic separation and for supercritical fluid chromatography is principally known to the person skilled in the art.

Preferred embodiments are shown in FIGS. 1 a), fig.1b) and fig.1c.

In one embodiment the isomers are collected at the outlet of the column. This is shown in FIG. 1a) in a schematic representation. Carbon dioxide is provided by a carbon dioxide cylinder 1. The alcohol 2 is added to the carbon dioxide stream and is transported to the chromatographic column 3 comprising the chiral stationary phase 4 by means of a SFC pump 5. The mixture 6 of chiral isomers of formula (I-A) or (I-B) is injected into the mobile phase by adequate injection means. The column 3 comprising the chiral stationary phase 4 is thermostatted by means of a heating device 7, being particular a SFC (column-) oven, the eluate 8 leaving the chromatographic column is analyzed/detected by a detection means 9, particularly a UV or diode array detector, being linked to a computer 10 controlling also the whole SFC equipment 11. The chromatographic conditions, particularly pressure, are controlled by a restrictor 12. When the eluate is passing the restrictor it is expanded and the supercritical carbon dioxide 13 is changing its aggregation state and transforms into gaseous carbon dioxide 14, yielding a remaining eluate and leaving the separated isomers 15, 15′ in the vessel 16, 16′. The flow of the remaining eluate is directed by the valve 17 into the first vessel 16 respectively second vessel 16′.

As a fact of the pressure release the supercritical carbon dioxide changes to gaseous carbon dioxide and the mobile phase changes dramatically its composition and solubility properties, hence, the chiral isomers separated tend to adhere to the tube 18 between the restrictor 12 and valve 17. These residues 19 tend to contaminate further fractions when they pass this site of chromatographic separation.

In order to overcome this problem a second embodiment of equipment is preferred. This modified equipment 11′ is identical to the equipment 1 just described except that between the detection means 9, particularly the diode array detector, and the restrictor 12 a T-junction 20 is localized by which a flow of flushing/rinsing solvent 21 is introduced into the flow of eluate by means of a pump 22, particularly an HPLC-pump. This flushing/rinsing solvent is rinsing also in the absence of the carbon dioxide continuously the tube 18 between the restrictor 12 and valve 17. As the flushing/rinsing solvent is a solvent which has a high solubility for the chiral isomers of formula (I-A) or (I-B) no residues are formed and, hence, no contamination of the further fractions occurs. Particularly suitable flushing/rinsing solvents are hydrocarbons, particularly heptane. In one, very preferred, embodiment the flushing/rinsing solvent is an alcohol, particularly the same alcohol which is part of the mobile phase, i.e. the alcohol being added to the carbon dioxide before entering the separation column, preferably ethanol or methanol.

By using an SMB-SFC, i.e. the SMB set up, such as disclosed in U.S. Pat. No. 5,518,625 and WO 03/051867 A1, for the supercritical fluid chromatography, the problem of residues formed after the restrictor does not exist as a result of a different experimental set up.

The step b) of the process of the invention leads to a separation of the mixture of isomers of formula (I-A) or (I-B). At least one of these isomers is separated out of the mixture. This isomer is preferably collected.

Therefore, the process comprises a step e) of collecting the desired isomer (I_{des}).

The non-desired isomer(s) either separated individually or as a residue may be isomerized.

Details for the isomerization can be found in WO 2012/152779 A1, the entire content of which is hereby incorporated by reference.

Therefore, in one embodiment, the chiral chromatographic separation of step b) yields a desired isomer (I_{des}) and a residual (I′) and further comprises the steps of
c) isomerizing the chirality at the center in the ring indicated by * in formula (I-A) or (I-B) of the isomers of the residual (I′) being separated in step b) yielding an isomerized product;
e) collecting the desired isomer (I_{des}).

Preferably, the isomerization of the chirality center(s) of compounds of formula (I-A) in step c) takes place by exposure of the residual (I′) to a temperature of above 150° C., particularly between 160 and 500° C. However, the temperatures should not be too high to avoid undesired degradation of the isomers. It has been found that a temperature between 160 and 300° C. gives good results.

In another embodiment the isomerization in step c) takes place by exposure of the residual (I′) to an acid of a pKₐ of smaller than 2, particularly smaller than 1. This method of isomerization is the preferred one.

This isomerization particularly changes the chirality at the chiral center of the position 2 only.

The isomerization in step c) leads to a change of the configuration at the center indicated by *, so that, after isomerization, the ratio of numbers of molecules in the R-configuration to the one in the S-configuration is about 50:50. It is clear to the person skilled in the art that real isomerization may differ from a ratio of 50:50 despite the isomerization is complete. Although complete isomeriztion is desired, also incomplete isomerizations are useful for the present invention as long as the amount of desired isomer is increased by the isomerization. It has been found that the ratio of the amount of desired isomer to amount of the non-desired isomer is at least 25:75, particularly at least 30:70, preferably at least 40:60 after the isomerization step.

The isomerized product can be introduced into the chromatographic separation of step b). Therefore, the process comprises preferably a further step d) of
d) introducing the isomerized product into the chromatographic separation of step b).

The isomerization allows converting undesired isomers, particularly isomers having a low or lower physiologically activity, into desired isomers, particularly into isomers having a higher physiologically activity.

It has been observed that it is particularly the R-configuration at the chiral centers marked by * in the formulae in this document are physiologically particularly active. This is for example shown by S. K. Jensen in Vitamins and Hormones 2007, Vol. 76, 281-308, the entire content of which is hereby incorporated by reference. Hence, it is preferred that the desired chiral isomers of formula (I-A) or (I-B) has the R-configuration at the carbons marked by *.

It is preferred that the desired isomer (I_{des}) is selected from the group consisting of

(2R, 4′R, 8′R)-α-tocopherol, (2R, 4′R, 8′R)-α-tocopheryl acetate, (2R, 4′R, 8′R)-β-tocopherol, (2R, 4′R, 8′R)-β-tocopheryl acetate, (2R, 4′R, 8′R)-γ-tocopherol, (2R, 4′R, 8′R)-γ-tocopheryl acetate, (2R, 4′R, 8′R)-δ-tocopherol and (2R, 4′R, 8′R)-δ-tocopheryl acetate;
(2R, 4′R, 8′R)-3,4-dehydro-α-tocopherol, (2R, 4′R, 8′R)-3,4-dehydro-α-tocopheryl acetate, (2R, 4′R, 8′R)-3,4-dehydro-β-tocopherol, (2R, 4′R, 8′R)-3,4-dehydro-β-tocopheryl acetate, (2R, 4′R, 8′R)-3,4-dehydro-γ-tocopherol, (2R, 4′R, 8′R)-3,4-dehydro-γ-tocopheryl acetate, (2R, 4′R, 8′R)-3,4-dehydro-δ-tocopherol and (2R, 4′R, 8′R)-3,4-dehydro-δ-tocopheryl acetate;
(2R)-α-tocotrienol, (2R)-α-tocotrienyl acetate, (2R)-β-tocotrienol, (2R)-β-tocotrienyl acetate, (2R)-γ-tocotrienol, (2R)-γ-tocotrienyl acetate, (2R)-δ-tocotrienol and (2R)-δ-tocotrienyl acetate;
   and preferably is selected from the group consisting of (2R, 4′R, 8′R)-α-tocopherol, (2R, 4′R, 8′R)-3,4-dehydro-α-tocopherol and (2R)-α-tocotrienol.

FIG. 1c) shows a schematic representation of such a preferable process. The mixture 6 of isomers of formula (I-A) provided in step a) are separated by means of supercritical fluid chromatography with supercritical carbon dioxide 13 as a mobile phase and an amylose tris(3,5-dimethylphenylcarbamate) coated or immobilized on a silica support as a chiral stationary phase 4 in step b). The chiral chromatographic separation of step b) yields a desired isomer (I_{des}) 23 and a residual (I′) 24. The residual (I′) 24 is isomerized in step c), so that the chirality at the center in the ring indicated by * in formula (I-A) or (I-B) of the isomers of the residual (I′) being separated in step b) yielding an isomerized product 25.

The isomerized product 25 is introduced in step d) into the chromatographic separation of step b) and the desired isomer (I_{des}) is collected in step e). Preferably this process is a continuous process, so that the isomerized product 25 is continuously fed to an incoming stream of mixture of isomers of formula (I-A) or (I-B) and the mobile phase at the entrance of the column in which the separation of step b) is performed. The collection of the desired isomer (I_{des}) is preferably also continuously collected.

This process is much preferred as it allows in a very cost efficient way to produce continuously the desired isomer (I_{des}) collected in step e), i.e. directly after the separation of desired isomer (I-A) or (I-B) and residual (I′) in step b).

It has been found that for a particularly good separation the chiral chromatographic separation uses Simulated Moving Bed (SMB) chromatography. Simulated Moving Bed (SMB) chromatography is a known method for separating racemic mixtures and is disclosed for example in U.S. Pat. No. 5,518,625 and WO 03/051867 A1, the entire contents of which is hereby incorporated by reference. The use of SMB set-up for supercritical fluid chromatography, i.e. SMB-SFC, is very advantageous if mixtures of chiral isomers of formula (I-A) or (I-B) are to be separated in large scale, particularly in industrial scale. For example, U.S. Pat. No. 5,770,088 and EP0687491 A1, the entire contents of which is hereby incorporated by reference, disclose SMB-SFC.

It is particularly useful for this application to use supercritical carbon dioxide due to the low viscosity and low density lead to small pressure drop in large respectively long columns. In this context it is important to mention that it has been found that the separation is better at low pressures, particularly near the critical pressure of 7.39 MPa. Furthermore, as already mentioned before, the problems of contamination by residues at the outlet of the equipment are inexistent in the SMB setup. All these points show that the combination of SMB and SFC are very advantageous.

Furthermore, isomer fractions collected by SFC-SMB are highly concentrated as no dilution is introduced to the sample mixture due to the fact that supercritical CO₂ is expanded to the gaseous state (normal pressure at the SMB-outlet) allowing collection of highly enriched and highly pure isomers only diluted by smaller amounts of modifier used for chiral separation.

The present invention allows the separation in a quantitative manner. It, furthermore, allows the separation of the isomer in a high isomeric purity of at least one of the desired isomer out of a mixture of chiral isomers of formula (I-A) or (I-B).

Particularly, the physiologically most active isomer(s), particularly (2R, 4′R, 8′R)-α-tocopherol, can be isolated.

It is important to realize that this separation is quantitative and high isomeric purities can be obtained.

On the one hand, the present process shows an extremely good separation of the isomers and on the other hand the method is extremely fast. It has been shown that this method is able to separate the chiral isomers in less than 5 minutes in an analytical scale and less than 9 minutes in semi-preparative scale. Due to the strongly reduced number of isomers to be separated the separation of (2-ambo)-tocopherols can be even separated within 5 minutes in an analytical as well as semi-preparative scale.

It has been observed that the majority of the peaks in the chromatographic separation which correspond to the individual chiral isomers are well base-line separated. Hence, a specific isolation of the isomers is possible, and a very high isomeric purity of the isolated chiral isomers can be achieved.

Finally, the method is very advantageous in that the mobile phase completely or at least mainly consists of carbon dioxide which is transferred from the supercritical aggregation state into the gaseous aggregation state on decompression. Hence, the product obtained at the end of the separation is in its pure form, respectively almost pure form, in case where solvents or further ingredients are part of the mobile phase. The separation of these is much easier as compared to their elimination in conventional solvent chromatographic separation techniques.

This absence of solvents, respectively the reduced amount of them, is very advantageous in view of speed and cost. Furthermore, compared to standard solvents, carbon dioxide is a relative cheap and easily recyclable medium and is inert and of low toxicity. Furthermore gaseous carbon dioxide continuously prevents the obtained product(s) from degradation/oxidation.

Carbon dioxide is a natural occurring chemical or a waste product of oxidation reactions of organic compound. Particularly, carbon dioxide is produced in huge quantities and has been found to be is responsible for the warming of the global climate. Hence, carbon dioxide produced in the carbon dioxide isolated or recuperated from natural or waste sources has a very positive eco-balance.

The use of supercritical dioxide as mobile phase of principle part of the mobile phase not only eliminates or reduces to a large extent the use of organic solvents in the separation.

Furthermore, carbon dioxide is available in almost unlimited quantities and is regarded as waste product.

All this leads to the fact that use of carbon dioxide is not only very advantageous in view of ecology and working safety but also in view of economy.

The up-scaling of the analytical respectively semi-preparative method to a quantitative, respectively industrial scale can be achieved by the person skilled in the art. This is particularly achieved by using the technique of the Simulated Moving Bed (SMB) chromatography.

I one embodiment after the separating of chiral isomers of formula (I-A) or (I-B) in which R² represents hydrogen is then reacted in a step f)
f) reacting the desired isomer (I-A) or (I-B) having R²═H with a protecting agent
to yield the isomer of formula (I-A) or (I-B) in which R² represents a phenol protection group.

Therefore, preferably (2R, 4′R, 8′R)-tocopherols, particularly (2R, 4′R, 8′R)-α-tocopherol, is as described above in detail, separated respectively collected as desired isomer (I_{des}) and reacted in step f) with a protecting agent, to yield (2R, 4′R, 8′R)-tocopherols, particularly (2R, 4′R, 8′R)-α-tocopherol, in its protected form, preferably (2R, 4′R, 8′R)-tocopheryl acetates, particularly (2R, 4′R, 8′R)-α-tocopheryl acetate.

In a further aspect, the invention relates to an use of supercritical fluid chromatography with supercritical carbon dioxide as a mobile phase and an amylose tris(3,5-dimethylphenylcarbamate) coated or immobilized on a silica support as a chiral stationary phase (CSP) for preparing a chromane or chromene which is selected from the group consisting of (2R, 4′R, 8′R)-α-tocopherol, (2R, 4′R, 8′R)-β-tocopherol, (2R, 4′R, 8′R)-γ-tocopherol, (2R, 4′R, 8′R)-δ-tocopherol; (2R, 4′R, 8′R)-3,4-dehydro-α-tocopherol, (2R, 4′R, 8′R)-3,4-dehydro-β-tocopherol, (2R, 4′R, 8′R)-γ-dehydro-y-tocopherol, (2R, 4′R, 8′R)-3,4-dehydro-δ-tocopherol, (2R)-α-tocotrienol, (2R)-β-tocotrienol, (2R)-γ-tocotrienol and (2R)-δ-tocotrienol;
particularly selected from the group consisting of (2R, 4′R, 8′R)-α-tocopherol, (2R, 4′R, 8′R)-3,4-dehydro-α-tocopherol and (2R)-α-tocotrienol,
   preferably (2R, 4′R, 8′R)-α-tocopherol, in a isomeric purity of more than 95% by weight.

In an even further aspect, the invention relates to a process of manufacturing a food or a feed or a food supplement or a feed supplement or a pharmaceutical composition comprising the steps of
i) preparing a compound of formula (I-A) or (I-B) having an isomeric purity of more than 95% by weight by a process as described above in detail;

ii) adding a compound of formula (I-A) or (I-B) of step i) to at least one food ingredient or at least one feed ingredient or at least one food supplement ingredient or at least one feed supplement ingredient or at least one ingredient for a pharmaceutical composition.

Therefore a food or a feed or a food supplement or a feed supplement or a pharmaceutical composition can be prepared by a process just described above.

### FIGURES

FIGS. 1a) and 1) show a schematic representation of an equipment for chiral chromatographic separation and for supercritical fluid chromatography. FIG. 1b) shows the schematic representation of an embodiment preferred over the one depicted in FIG. 1a) where the equipment is rinsed with a solvent. FIG. 1c) shows schematically a process which comprises an isomerization step.

### LIST OF REFERENCE SIGNS

1 carbon dioxide cylinder
2 alcohol
3 chromatographic column
4 chiral stationary phase
5 SFC pump
6 mixture of isomers of formula (I-A)or (I-B)
7 heating device, SFC oven
8 eluate
9 detection means, diode array detector
10 computer
11 SFC equipment
11′ modified SFC equipment
12 restrictor
13 supercritical carbon dioxide
14 gaseous carbon dioxide
15,15′ separated isomers
16,16′ vessels, first vessel, second vessel
17 valve
18 tube between restrictor 12 and valve 17
19 residues
20 T-junction
21 Flushing/rinsing solvent
23 pump
desired isomer (I_{des})
24 residual (I′)
25 isomerized product

### EXAMPLES

The present invention is further illustrated by the following experiments.

### 1. Chromatographic Separation

Starting Materials:

Solvents and reagents used as received were methanol (Merck Lichrosolv Reag Ph Eur, gradient grade for liquid chromatography, order no. 1.06007.2500), n-heptane (Merck Lichrosolv, for liquid chromatography, order no. 1.04390.1000), CO₂ (Quality 4.8, Carbagas, Schweiz).

Chromatography:

The separations were performed on an Agilent Technologies 1260 Infinity Analytical SFC System comprising an Aurora SFC fusion A5 module, SFC binary pump (model G 4302A), Degasser, SFC autosampler, DAD (Diode Array Detector) (DAD SL, model G 1315C), Thermostatted Column Compartment and SFC Accessory Kit.

The DAD-detection range used and collected was 190-500 nm. Depending on the concentrations the signal at 210 nm, 290 nm, 295 nm has been used in the following.

The resulting chromatograms are shown in FIGS. 2 to 11. The x-axis of the chromatograms represents the retention time (tᵣₑₜ) in minutes. The y-axis of the chromatograms represents the absorbance (A) in arbitrary units (AU resp. mAU) by which the isomer distribution is detected.

Separation of (all-rac)-α-tocopherol

### Example 1

A 50% by weight solution of (all-rac)-α-tocopherol (DSM Nutritional Products) in n-heptane was injected and separated on a chiral stationary phase (Daicel Chiralpak® AD-3, 250 mm×4.6 mm; eluent: supercritical CO₂/10% by volume methanol, 40° C., 150 bar back pressure; flow 3.0 ml/min; detection 295 nm, 5 μl injection).

FIG. 2a) shows the obtained chromatogram of (all-rac)-α-tocopherol. From the 8 isomers of (all-rac)-α-tocopherol 3 isomers have been separated in a good baseline separation (3.57 min., 3.70 min. and 4.01 min). The peak with retention time at maximum of 3.70 min. could be identified as (2R, 4′R, 8′R)-α-tocopherol by a control experiment with a reference sample of (2R, 4′R, 8′R)-α-tocopherol with the same column and the same conditions. The chromatogram of this reference (2R, 4′R, 8′R)-α-tocopherol is shown in FIG. 2b).

Example 1 shows that the (all-rac)-α-tocopherol can be separated in less than 5 minutes by separation with supercritical fluid chromatography with supercritical carbon dioxide as a mobile phase and an amylose tris(3,5-dimethylphenyl-carbamate) coated or immobilized on a silica support as a chiral stationary phase (CSP). It further shows that the (2R, 4′R, 8′R)-α-tocopherol is a base-separated peak which can be easily isolated.

Separation of (all-rac)-α-tocopherol and 2-ambo-α-tocopherol by Two Columns in Sequence

### Example 2

A solution of (all-rac)-α-tocopherol (DSM Nutritional Products) (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 210 nm, 5 μl injection).

FIG. 3a) shows the obtained chromatogram of (all-rac)-α-tocopherol.

Furthermore, a solution of (2-ambo)-α-tocopherol (DSM Nutritional Products) (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 210 nm, 5 μl injection).

FIG. 3b) shows the obtained chromatogram of 2-ambo-α-tocopherol.

From the 8 isomers of (all-rac)-α-tocopherol 4 isomers have been separated in a good baseline separation (tᵣₑₜ=9.56 min., 8.78 min., 8.52 min. and 8.17 min). Both isomers of 2-ambo-α-tocopherol have been separated in a good baseline separation (tᵣₑₜ=8.78 min. and 7.86 min.).

The peak with retention time at maximum of 8.78 min. of (all-rac)-α-tocopherol and 2-ambo-α-tocopherol could be identified as (2R, 4′R, 8′R)-α-tocopherol by a control experiment with a reference sample of (2R, 4′R, 8′R)-α-tocopherol with the same column and same conditions. The resulting chromatogram of this reference (2R, 4′R, 8′R)α-tocopherol is shown in FIG. 3c).

The peak with retention time at maximum of 7.85 min. of 2-ambo-α-tocopherol could be, hence, identified as (2S, 4′R, 8′R)-α-tocopherol.

Compared to the experiment of example 1, the data of example 2 show that the coupling of 2 columns and use lower back pressure leads to an even better peak separation and the separation time is only extended to a minor extent, i.e. the whole chromatographic separation is achieved within a timeframe of less than 10 minutes.

### Example 3

A solution of (all-rac)-α-tocopherol (DSM Nutritional Products) (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 35° C., 110 bar back pressure; flow 4.0 ml/min; detection 295 nm, 5 μl injection).

FIG. 4a) shows the obtained chromatogram of (all-rac)-α-tocopherol. Furthermore, a solution of (2-ambo)-α-tocopherol (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 35° C., 110 bar back pressure; flow 4.0 ml/min; detection 295 nm, 5 μl injection).

FIG. 4b) shows the obtained chromatogram of (2-ambo)-α-tocopherol.

From the 8 isomers of (all-rac)-α-tocopherol 4 isomers have been separated in a good baseline separation (tᵣₑₜ=6.65 min., 6.05 min., 5.81 min. and 5.52 min). Both isomers of (2-ambo)-α-tocopherol have been separated in a good baseline separation (tᵣₑₜ=6.05 min. and 5.29 min.).

The peak with retention time at maximum of 6.05 min. of (all-rac)-α-tocopherol and (2-ambo)-α-tocopherol could be identified as (2R, 4′R, 8′R)-α-tocopherol by a control experiment with a reference sample of (2R, 4′R, 8′R)-α-tocopherol with the same column and same conditions. The chromatogram of this reference (2R, 4′R, 8′R)α-tocopherol is shown in FIG. 4c).

The peak with retention time at maximum of 5.29 min. of (2-ambo)-α-tocopherol could be, hence, identified as (2S, 4′R, 8′R)-α-tocopherol.

Compared to the experiment of example 2, the data of example 3 show that despite the higher pressure used when lowering the temperature, a still excellent separation results at a very fast separation time of less than 7 minutes for the whole chromatographic separation.

Separation of (all-rac)-γ-tocopherol and 2-ambo-γ-tocopherol by Two Columns in Sequence

### Example 4

A solution of (all-rac)-γ-tocopherol (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 210 nm, 15 μl injection).

FIG. 5a) shows the obtained chromatogram of (all-rac)-γ-tocopherol.

Furthermore, a solution of 2-ambo-γ-tocopherol (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 210 nm, 15 μl injection).

FIG. 5b) shows the obtained chromatogram of (2-ambo)-γ-tocopherol.

In case of (all-rac)-γ-tocopherol 3 peaks have been separated in a good baseline separation (tᵣₑₜ=8.39 min., 8.85 min. and 9.36 min) (FIG. 5a).

Both isomers of (2-ambo)γ-tocopherol have been separated in a good baseline separation (tᵣₑₜ=8.86 min. and 9.36 min) (FIG. 5b).

The peak with retention time at maximum of 9.36 min. of (2-ambo)-γ-tocopherol could be identified as (2R, 4′R, 8′R)-γ-tocopherol by a control experiment with a reference sample of (2R, 4′R, 8′R)-γ-tocopherol with the same column and same conditions. The chromatogram of this reference (2R, 4′R, 8′R)-γ-tocopherol is shown in FIG. 5c).

The peak with retention time at maximum of 8.86 min. of (2-ambo)-γ-tocopherol could be, hence, identified as (2S, 4′R, 8′R)-γ-tocopherol.

Separation of (all-rac)-3,4-dehydro-α-tocopherol and (2-ambo)-3,4-dehydro-α-tocopherol by Two Columns in Sequence

### Example 5

A solution of (all-rac)-3,4-dehydro-α-tocopherol (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 295 nm, 15 μl injection).

FIG. 6a) shows the obtained chromatogram of (all-rac)-3,4-dehydro-α-tocopherol.

Furthermore, a solution of (2-ambo)-3,4-dehydro-α-tocopherol (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 295 nm, 15 μl injection).

FIG. 6b) shows the obtained chromatogram of (2-ambo)-3,4-dehydro-α-tocopherol.

From the 8 isomers of (all-rac)-3,4-dehydro-α-tocopherol 3 isomers have been separated in a good baseline separation (tᵣₑₜ=6.60 min., 7.15 min. and 7.82 min). Furthermore, overlapping peaks leading to local maximums at tᵣₑₜ=6.81 min, 6.92 min. and 8.07 min. could been observed (FIG. 6a).

Both isomers of (2-ambo)-3,4-dehydro-α-tocopherol have been separated in a good baseline separation (tᵣₑₜ=6.60 min. and 7.82 min.)(FIG. 6b).

The peak with retention time at maximum of 7.82 min. of (all-rac)-3,4-dehydro-α-tocopherol and (2-ambo)-3,4-dehydro-α-tocopherol could be identified as (2R, 4′R, 8′R)-3,4-dehydro-α-tocopherol by a control experiment with a reference sample of (2R, 4′R, 8′R)-3,4-dehydro-α-tocopherol with the same column and same conditions.

The resulting chromatogram of this reference (2R, 4′R, 8′R)-3,4-dehydro-α-tocopherol is shown in FIG. 6c).

The peak with retention time at maximum of 6.60 min. of (2-ambo)-3,4-dehydro-α-tocopherol and (all-rac)-3,4-dehydro-α-tocopherol could be, hence, identified as (2S, 4′R, 8′R)-3,4-dehydro-α-tocopherol.

Separation of (rac)-α-tocotrienol by Two Columns in Sequence

### Example 6

A solution of (rac)-α-tocotrienol (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 295 nm, 15 μl injection).

FIG. 7a) shows the obtained chromatogram of (rac)-α-tocotrienol.

The 2 isomers (=enantiomers) of-(rac)-α-tocotrienol could be observed as two slightly overlapping peaks (tᵣₑₜ=10.68 min. and 10.88 min.)(FIG. 7a).

The peak with retention time at maximum of 10.68 min. of (rac)-α-tocotrienol could be identified as (2R)-α-tocotrienol by a control experiment with a reference sample of (2R)-α-tocotrienol with the same column and same conditions.

The resulting chromatogram of this reference (2R)-α-tocotrienol is shown in FIG. 7b). The peak with retention time at maximum of 10.88 min. of 2-ambo-α-tocotrienol could be, hence, identified as (2S)-α-tocotrienol.

Separation of (rac)-γ-tocotrienol by Two Columns in Sequence

### Example 7

A solution of (rac)-γ-tocotrienol (10 mg) in n-heptane (10 ml) was prepared, injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3.0 ml/min; detection 295 nm, 15 μl injection).

FIG. 8a) shows the obtained chromatogram of (rac)-γ-tocotrienol.

The 2 isomers of (rac)-γ-tocotrienol could be separated in a good baseline separation (tᵣₑₜ=12.49 min. and 13.15 min.)(FIG. 8a).

The peak with retention time at maximum of 12.49 min. of (rac)-γ-tocotrienol could be identified as (2R)-γ-tocotrienol by a control experiment with a reference sample of (2R)-γ-tocotrienol with the same column and same conditions.

The resulting chromatogram of this reference (2R)-γ-tocotrienol is shown in FIG. 8b). The peak with retention time at maximum of 13.15 min. of (rac)-γ-tocotrienol could be, hence, identified as (2S)-γ-tocotrienol.

Separation of (2-ambo)-α-tocopherol in semi-preparative scale

### Example 8

### (No Rinsing/Flushing)

A 50% by weight solution of (2-ambo)-α-tocopherol in n-heptane was injected and separated on a chiral stationary phase (Daicel Chiralpak® AD-3, 250 mm×4.6 mm, 2 columns in sequence; eluent: supercritical CO₂/10% by volume methanol, 40° C., 90 bar back pressure; flow 3 ml/min; detection 290nm, 5 μl injection).

FIG. 9a) shows the chromatogram of (2-ambo)-α-tocopherol. As the amount of material separated is about a factor a factor 500 higher as in example 2, the peaks in the chromatogram are broader, however, are still separated.

The product leaving the outlet of the column was collected in a first glass vessel. At the detection of the local minimum at the retention time the valve at the outlet of the column was turned so that the substance eluted after the switch was collected in the second glass vessel.

The content of each glass vessel was dissolved in methanol to yield an analytical solution ready for injection. The solutions were injected again in the same manner as shown above (conditions of example 2, however, another column lot have been used which lead to the fact that the peaks elute a slightly earlier than in Example 2) with an injection of 5 μl injection for analysis of identification and analysis of purity of the collected fractions.

The chromatogram of the first vessel is shown in FIG. 9b) and the chromatogram of the second vessel is shown in FIG. 9c)

These chromatograms show that the first glass vessel is pure (2S, 4′R, 8′R)-α-tocopherol (RRR-isomer is not detectable) whereas in the second glass vessel was (2R, 4′R, 8′R)-α-tocopherol and a small amount of (2S, 4′R, 8′R)-α-tocopherol. The isomeric purity of the (2R, 4′R, 8′R)-α-tocopherol was calculated from the area in the chromatogram (FIG. 9c) to be 89%.

### Example 9

### Rinsing/Flushing by N-Heptane

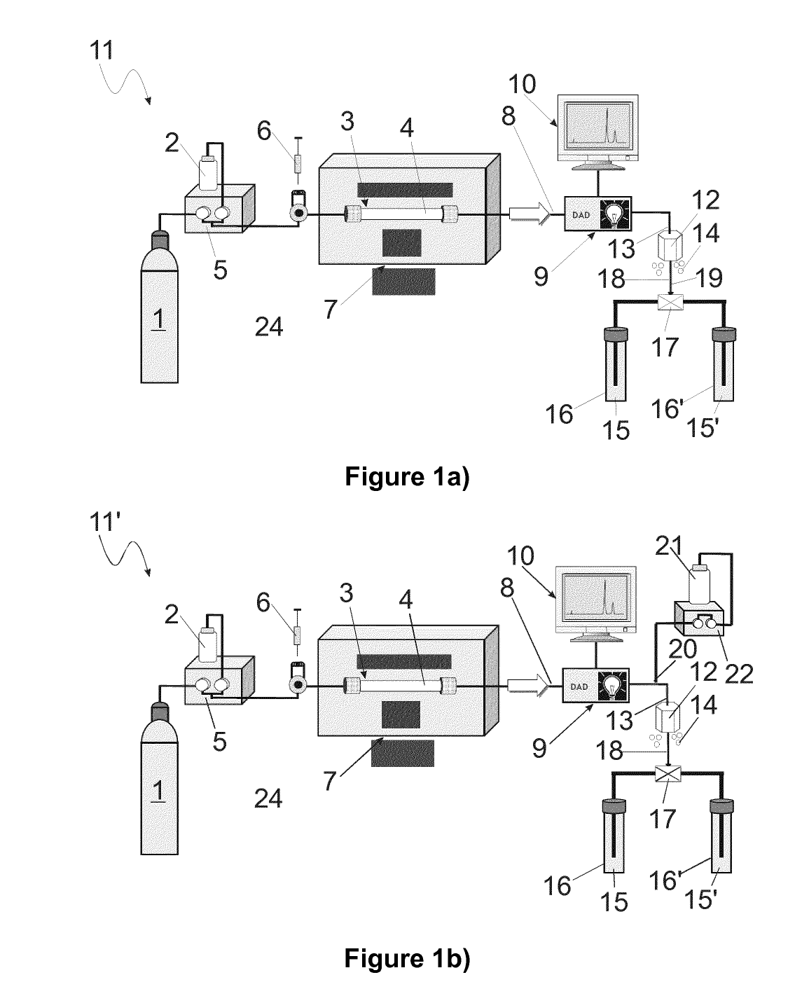

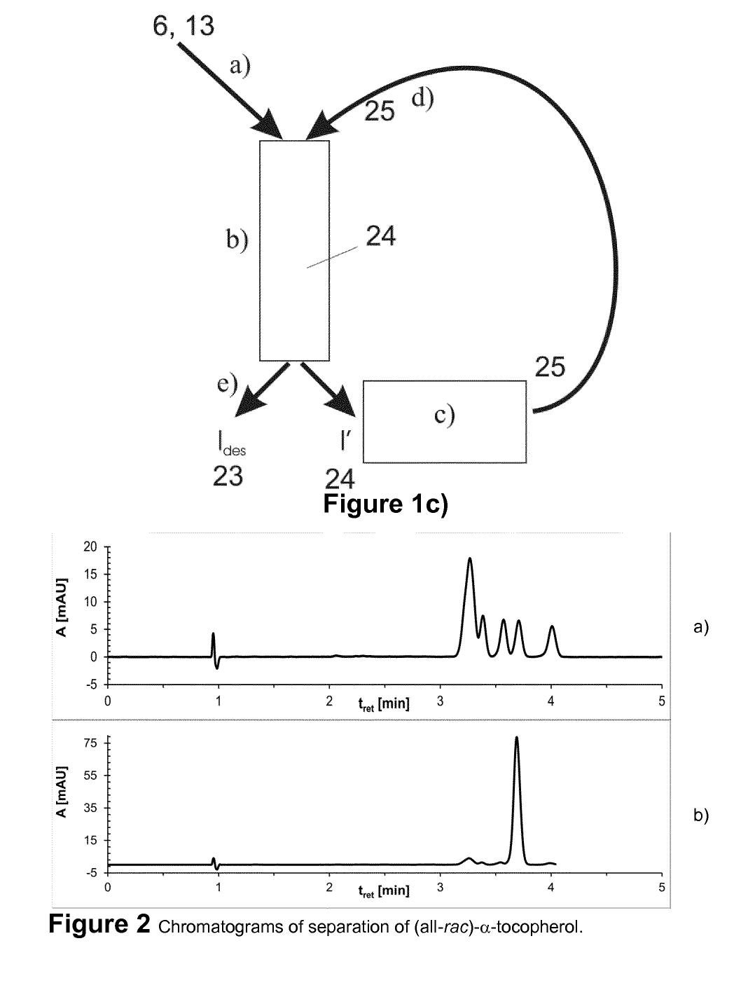

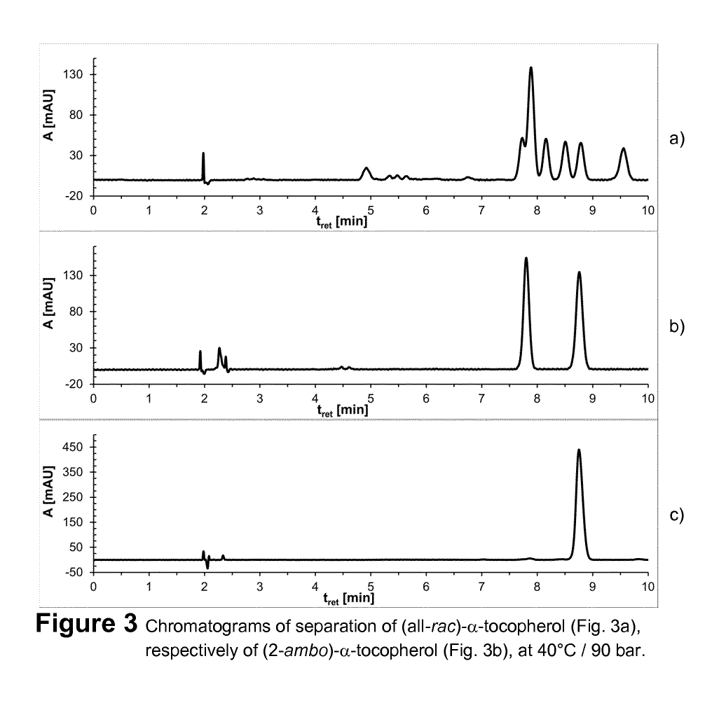

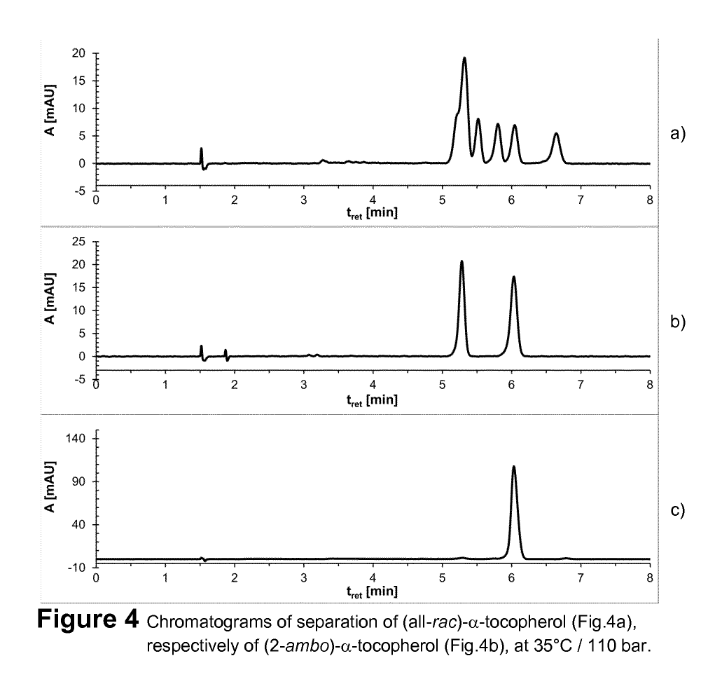

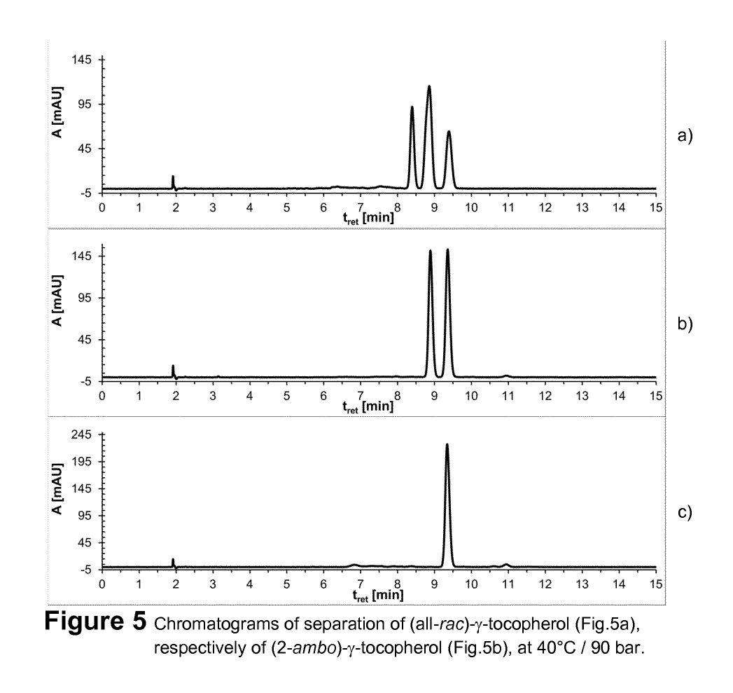

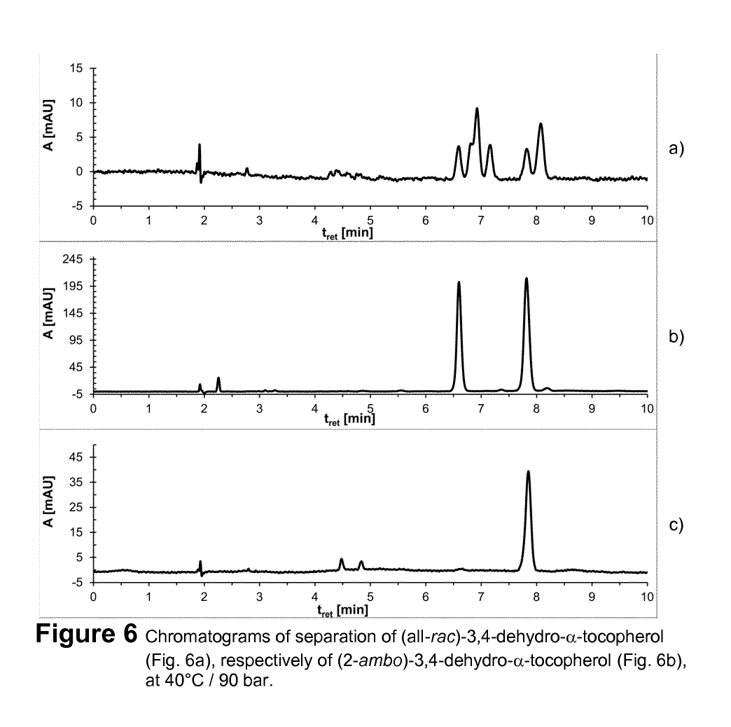

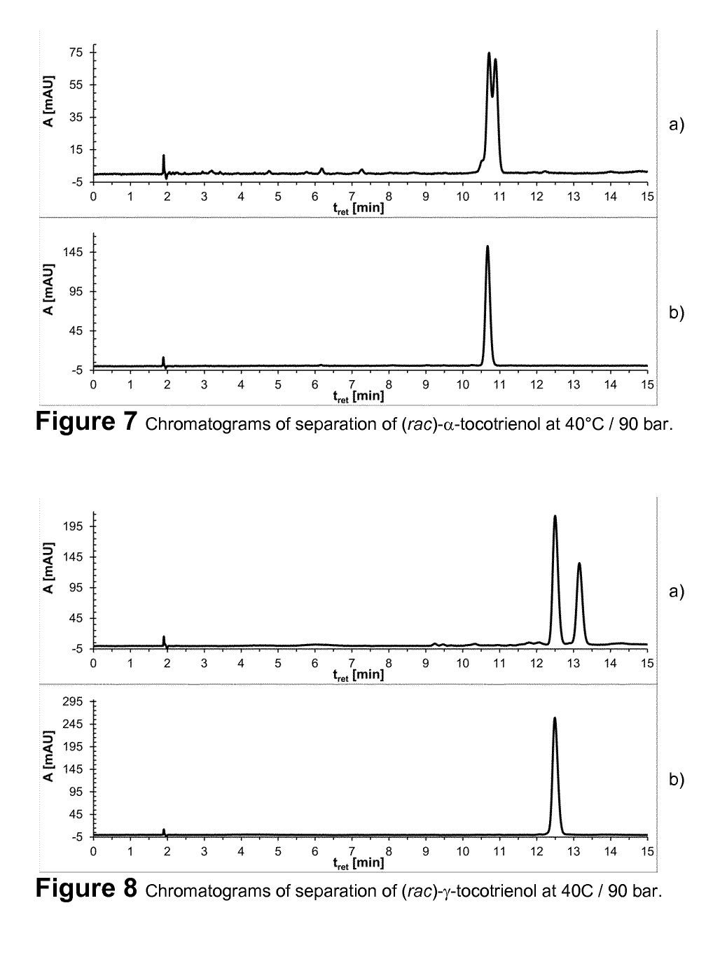

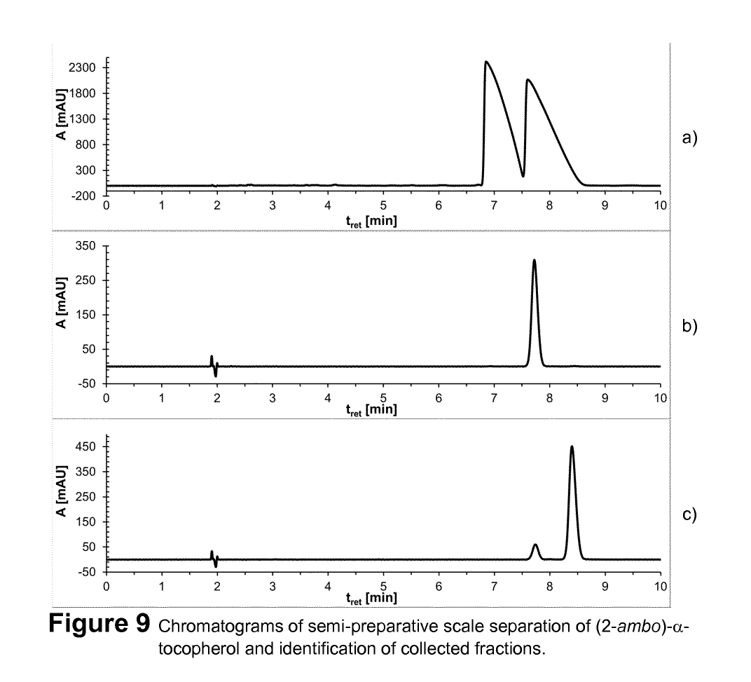

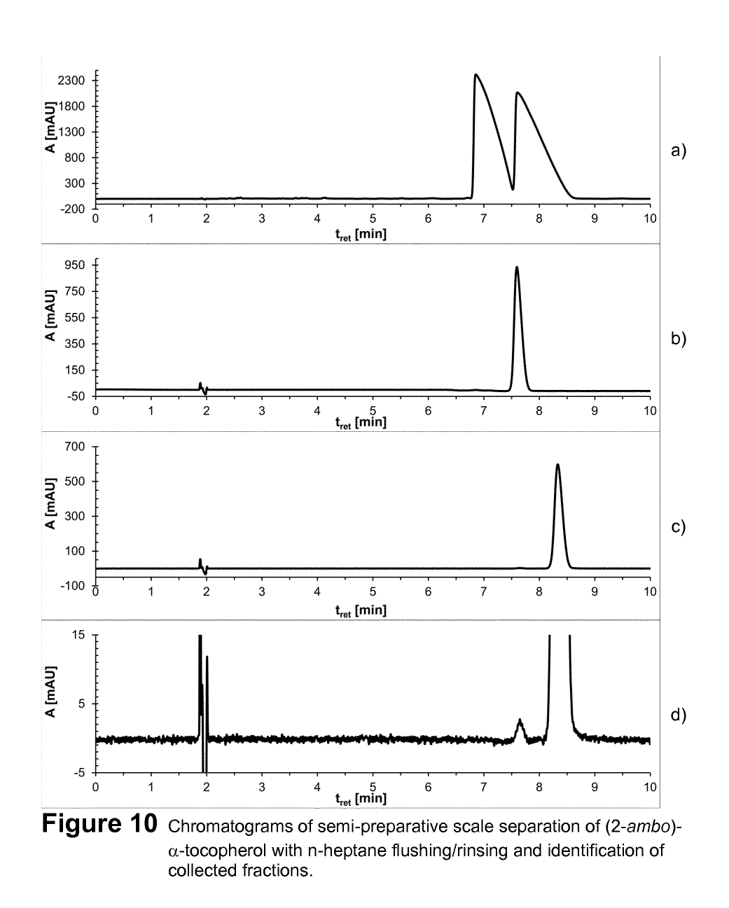

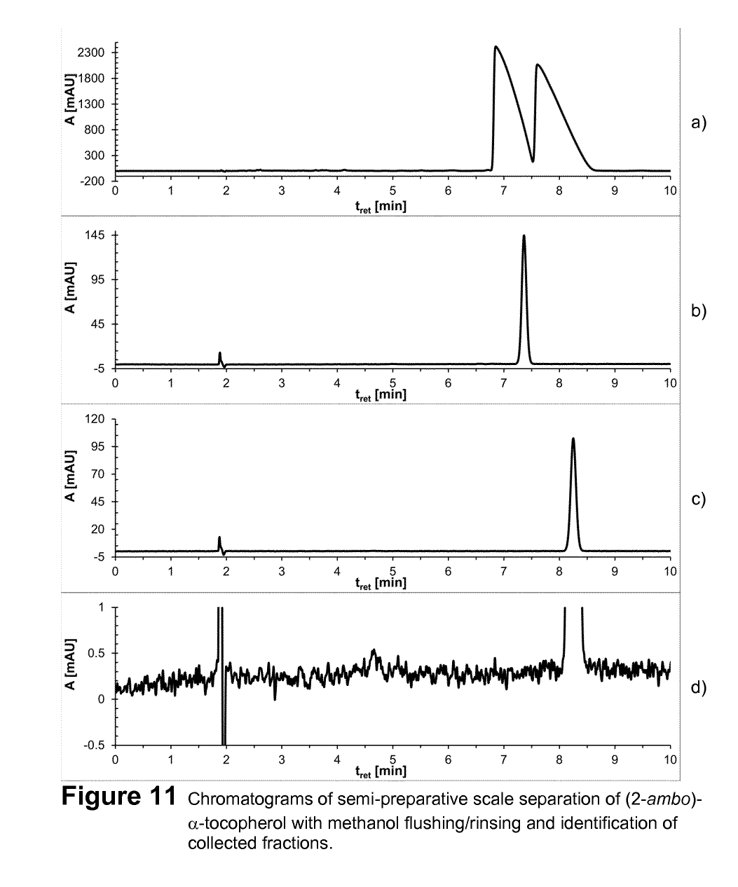

The example 9 was treated and measured identically to the example 8 except that between the detector and the restrictor a continuous flow (1 ml/min) of n-heptane was provided by means of a modular (stand-alone) HPLC pump according to the schematic diagram shown in FIG. 1b).

FIG. 10a) shows again the chromatogram during the high quantity separation.

FIG. 10b) shows the chromatogram of the sample from the first glass vessel. It could be identified to be pure (2S, 4′R, 8′R)-α-tocopherol (RRR is again not detectable).

FIG. 10c) shows the chromatogram of the sample from the second glass vessel. It shows that this sample is almost pure. Very pure (2R, 4′R, 8′R)-α-tocopherol with minor traces of fraction (2S, 4′R, 8′R)-α-tocopherol was collected. FIG. 10d) shows the chromatogram of FIG. 10c) in a representation where the y-axis is highly magnified. The isomeric purity of the (2R, 4′R, 8′R)-α-tocopherol was calculated from the area in the chromatogram (FIG. 10c, FIG. 10d) to be 99.7%.

### Example 10

### Rinsing/Flushing by Methanol

The example 10 was treated and measured identically to the example 9 except that methanol was used at flushing/rinsing solvent as well as solvent being part of the mobile phase, i.e. the alcohol being added to the carbon dioxide before entering the separation column. FIG. 11a) shows the chromatogram during the high quantity separation.

FIG. 11b) shows the chromatogram of the sample from the first glass vessel. It could be identified to be pure (2S, 4′R, 8′R)-α-tocopherol (RRR isomer is again not detectable).

FIG. 11c) shows the chromatogram of the sample from the second glass vessel. It shows that this sample is highly pure (2R, 4′R, 8′R)-α-tocopherol. No traces of (2S, 4′R, 8′R)-α-tocopherol could be detected as FIG. 11d) (being the chromatogram of FIG. 11c) in a representation where the y-axis is highly magnified) clearly shows.

Therefore, this example 4 shows that the disclosed process allows that, under optimal conditions, (2R, 4′R, 8′R)-α-tocopherol as well as (2S, 4′R, 8′R)-α-tocopherol can be obtained as absolutely isomerically pure (no other isomers detectable!) isomers from mixtures of said isomers.

1. Process of separating chiral isomers of chromane or chromene compounds of formula (I-A) or (I-B) 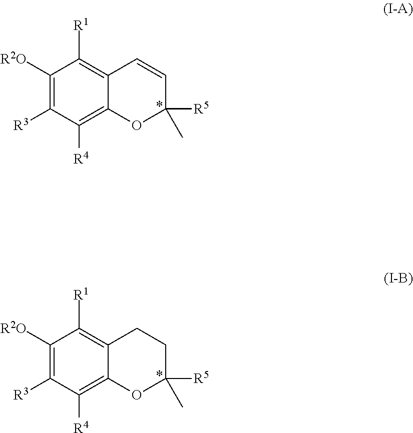
wherein R¹, R³ and R⁴ are independently from each other hydrogen or methyl groups;
R² represents hydrogen or a phenol protection group;
R⁵ represents either a linear or branched completely saturated C₆₋₂₅-alkyl group or a linear or branched C₆₋₂₅-alkyl group comprising at least one carbon-carbon double bond;
and wherein * represents a chiral center;
comprising the steps of
a) providing a mixture of isomers of formula (I-A) or (I-B) having different chiral configuration at the chiral centers represented by * in formula (I-A) or (I-B);
b) chiral chromatographic separation of the mixture of isomers of step a) by means of supercritical fluid chromatography with supercritical carbon dioxide as a mobile phase and an amylose tris(3,5-dimethylphenylcarbamate) coated or immobilized on a silica support as a chiral stationary phase (CSP).

2. Process according to claim 1 wherein R¹═R³═R⁴═CH₃.

3. Process according to claim 1, wherein R⁵ is of formula (III) 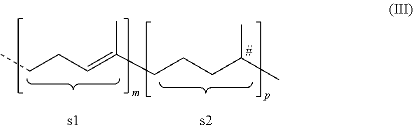
wherein m and p stand independently from each other for a value of 0 to 5 provided that the sum of m and p is 1 to 5,
and where the substructures in formula (III) represented by s1 and s2 can be in any sequence;
and the dotted line represents the bond by which the substituent of formula (III) is bound to the rest of formula (I-A) or (I-B);
and wherein # represents a chiral center, obviously except in case where said center is linked to two methyl groups.

4. Process according to claim 1 wherein R⁵ is of formula (III-A), particularly (III-ARR), or (III-B) 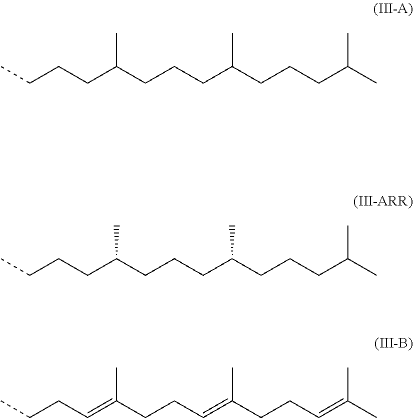
wherein the dotted line represents the bond by which the substituent of formula (III-A) or (III-B) is bound to the rest of formula (I-A) or (I-B);
and wherein # represents a chiral center.

5. Process according to claim 1, wherein the mobile phase comprises an alcohol particularly an alcohol being selected from the group consisting of methanol, ethanol, 1-propanol, 2-propanol, 1-butanol, 2-butanol, 2-methyl-1-propanol and 2-methyl-2-propanol.

6. Process according to claim 5 wherein the alcohol is used in a volume ratio of 1 to 30%, particularly of 1 to 25%, preferably of 5 to 20%, more preferably of 8 to 15%, relative to the supercritical carbon dioxide.

7. Process according to claim 1, wherein the chromatographic separation is done at a temperature in the range of between 31.3° C. and 80° C., preferably in the range of between 32° C. and 50° C., particularly between 35° C. and 45° C.

8. Process according to claim 1, wherein the chromatographic separation is done at a pressure in the range of between 73.9 to 200 bar, particularly between 74 to 150 bar, preferably between 75 and 100 bar, more preferably between 75 and 90 bar.

9. Process according to claim 1, wherein the chiral chromatographic separation of step b) yields a desired isomer (I_{des}) and a residual (I′) and further comprises the steps of
c) isomerizing the chirality at the center in the ring indicated by * in formula (I-A) or (I-B) of the isomers of the residual (I′) being separated in step b) yielding an isomerized product;
e) collecting the desired isomer (I_{des}).

10. Process according to step 9 wherein the process comprises a further step of
d) introducing the isomerized product into the chromatographic separation of step b).

11. Process according to claim 1, wherein the mixture of chiral isomers of formula (I-B) is (all-rac)-tocopherol, particularly (all-rac)-α-tocopherol.

12. Process according to claim 1, wherein the mixture of chiral isomers of formula (I-B) is (2-ambo)-tocopherol, particularly (2-ambo)-α-tocopherol.

13. Process according to claim 1 wherein the chiral chromatographic separation uses Simulated Moving Bed (SMB) chromatography.

14. Use of supercritical fluid chromatography with supercritical carbon dioxide as a mobile phase and an amylose tris(3,5-dimethylphenylcarbamate) coated or immobilized on a silica support as a chiral stationary phase (CSP) for preparing a chromane or chromene which is selected from the group consisting of (2R, 4′R, 8′R)-α-tocopherol, (2R, 4′R, 8′R)-β-tocopherol, (2R, 4′R, 8′R)-γ-tocopherol, (2R, 4′R, 8′R)-δ-tocopherol; (2R, 4′R, 8′R)-3,4-dehydro-α-tocopherol, (2R, 4′R, 8′R)-3,4-dehydro-β-tocopherol, (2R, 4′R, 8′R)-3,4-dehydro-γ-tocopherol, (2R, 4′R, 8′R)-3,4-dehydro-δ-tocopherol, (2R)-α-tocotrienol, (2R)-β-tocotrienol, (2R)-γ-tocotrienol and (2R)-γ-tocotrienol; particularly selected from the group consisting of (2R, 4′R, 8′R)-α-tocopherol, (2R, 4′R, 8′R)-3,4-dehydro-α-tocopherol and (2R)-α-tocotrienol, in an isomeric purity of more than 95% by weight.

15. Process of manufacturing a food or a feed or a food supplement or a feed supplement or a pharmaceutical composition comprising the steps of
i) preparing a compound of formula (I-A) or (I-B) having an isomeric purity of more than 95% by weight by a process according to claim 1; 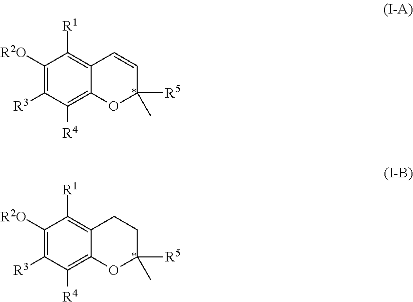
wherein R¹, R³ and R⁴ are independently from each other hydrogen or methyl groups;
R² represents hydrogen or a phenol protection group;
R⁵ represents either a linear or branched completely saturated C₆₋₂₅-alkyl group or a linear or branched C₆₋₂₅-alkyl group comprising at least one carbon-carbon double bond;
and wherein * represents a chiral center;
ii) adding a compound of formula (I-A) or (I-B) of step i) to at least one food ingredient or at least one feed ingredient or at least one food supplement ingredient or at least one feed supplement ingredient or at least one ingredient for a pharmaceutical composition.
